(19) United States
(12) Patent Application Publication
(10) Pub. No.: US 2003/0180780 A1
(43) Pub. Date: Sep. 25, 2003

(54) Stabilized inorganic particles

(76) Inventors: Jun Feng, Davis, CA (US); Bruce D. Hammock, Davis, CA (US); Ian M. Kennedy, Davis, CA (US); Goumin Shan, Carmel, IN (US); Angel Maquieira, Valencia (ES)

Correspondence Address: FENWICK & WEST LLP, SILICON VALLEY CENTER, 801 CALIFORNIA STREET, MOUNTAIN VIEW, CA, 94041, US

(21) Appl. No.: 10/393,702

(22) Filed: Mar. 19, 2003

Related U.S. Application Data
(60) Provisional application No. 60/365,845, filed on Mar. 19, 2002.

(51) Int. Cl. C12Q 1/68; G01N 33/53; G01N 33/551

(52) U.S. Cl. 435/6; 436/524; 435/7.1

(57) ABSTRACT

Silane-coated metal oxide nanoparticles having desirable optical properties are provided by the invention, along with methods for their preparation and use. The nanoparticles have improved chemical and physical stability and may be used as labeling reagents for biological and other molecules. The compositions may be prepared by contacting the metal oxide nanoparticle with a silane, and irradiating the mixture with microwave radiation.

### CROSS REFERENCE TO RELATED APPLICATIONS

This application claims the benefit of U.S. Provisional Application No. 60/365,845, filed Mar. 19, 2002, which is hereby incorporated in its entirety by reference.

### STATEMENT REGARDING FEDERALLY SPONSORED RESEARCH

This invention was made with government support under 5P42ES04699 awarded by the National Institute of Environmental Health Sciences and under DBI-0102662 awarded by the National Science Foundation. The government has certain rights in the invention.

### BACKGROUND

1. Field of the Invention

This invention relates to the fields of chemistry and biology.

2. Background of the Invention

Fluorescence is a widely used tool in chemistry and biological science. Fluorescent labeling of molecules is a standard technique in biology [31]. The labels are often organic dyes that give rise to the usual problems of broad spectral features, short lifetime, photobleaching, and potential toxicity to cells. The recent emerging technology of quantum dots has spawned a new era for the development of fluorescent labels using inorganic complexes or particles. These materials offer substantial advantages over organic dyes including larger Stokes shift, longer emission half-life, narrow emission peak and minimal photo-bleaching. However, quantum dot technology still is in its infancy, and is plagued by many problems including difficulties associated with reproducible manufacture, coating, and derivatization of quantum dot materials.

In addition, although the quantum yield of an individual quantum dot is high, the actual fluorescence intensity of each tiny dot is low. Grouping multiple quantum dots into larger particles is one approach for increasing the fluorescence intensity, but this nascent technology still suffers from drawbacks including difficulties in generating and maintaining uniform particle size distributions. Wider application of quantum dot technology therefore has been limited by the difficulties referred to above.

Alternative labels may be based on lanthanide-derived phosphors. Rare-earth metal elements such as europium are known for their unique optical (fluorescent/phosphorescent) properties. When their salts are dissolved in water, their fluorescence is quenched. Thus, many investigators have used europium and other rare-earth chelates to label biological molecules for the sensitive detection of proteins and nucleic acids [1], to carry out time-resolved fluorometric assays [2-7], and as labels in immunoassays [8-16]. However, this chelation chemistry often is expensive and complex, and so application of rare-earth chelation technology also has been limited to date.

Recently, nanoparticles have received much attention in biology [17-24]. These particles can have strong fluorescence that exhibits a spectrally sharp emission peak, large Stokes shift, and less quenching influence by other chemicals. Nanoparticles such as Eu₂O₃ particles also have been recognized as offering tremendous potential in obtaining large enhancement of emission intensity [25-26]. However, Eu₂O₃ and other nanoparticles are easily dissolved by acid during activation and conjugation, thereby losing their desirable properties. In addition, nanoparticles lack reactive groups that allow them to be derivatized easily and linked to analytes and other reagents, thus increasing the difficulty associated with using nanoparticles as labeling reagents for the study of biological and other molecules.

Silica and alumina surfaces have wide-ranging surface reactivities [27]; in particular, silica can be used as a cap to keep europium oxide from dissolving in acid in the conjugation process. However, coating with silica and alumina may increase the particle size, thereby compromising the advantageous properties of nanoparticles that render them suitable as labeling reagents.

The present invention addresses these and other limitations of the prior art by providing methods for stabilizing nanoparticles, and stabilized nanoparticle compositions that retain many or all of native particles' optical properties and enable the use of the stabilized nanoparticle to derivatize and so label biological and other materials.

### SUMMARY OF THE INVENTION

In one aspect the invention provides for stabilized nanoparticle compositions comprising a metal oxide particle having a desirable optical property coated with a silane. Preferred particle diameters are in the range of between about 10 and 1000 nm, more preferably between about 10 and 200 nm or between about 10 and 100 nm, and even more preferably between about 20 and 50 nm. The metal oxide particles have the generic formula MeₓO_{y}, wherein 1≦x≦2 and 1≦y≦3 and wherein preferably x=2 and y=3, and wherein preferably, Me is a rare earth element, a lanthanide (atomic number, z,=57 to 71) or an actinide metal (z=89 to 105). In preferred embodiments, Me is selected from the lanthanide series and includes, but is not limited to, europium (Eu), cerium (Ce), neodymium (Nd), samarium (Sm), terbium (Tb), dysprosium (Dy), gadolinium (Gd), holmium (Ho), or thulium (Tm), or Me may be chromium (Cr), yttrium (Y), or iron (Fe). Other suitable metal oxide particles include silicon oxide (SiO₂), aluminum oxide (Al₂O₃), titanium oxide (TiO₂), and zirconium oxide (ZrO₂) that are mixed with Eu₂O₃ or Eu³⁺.

In other preferred embodiments, the metal oxide particle comprises a doped metal oxide particle by which is meant a metal oxide, and a dopant comprised of one or more rare earth elements. Suitable metal oxides include, but are not limited to, yttrium oxide (Y₂O₃), zirconium oxide (ZrO₂), zinc oxide (ZnO), copper oxide (CuO or Cu₂O), gadolinium oxide (Gd₂O₃), praseodymium oxide (Pr₂O₃), lanthanum oxide (La₂O₃), and alloys thereof. The rare earth element comprises an element selected from the lanthanide series and includes, but is not limited to, europium (Eu), cerium (Ce), neodymium (Nd), samarium (Sm), terbium (Tb), dysprosium (Dy), gadolinium (Gd), holmium (Ho), thulium (Tm), an oxide thereof, and a combination thereof. In these preferred embodiments, the desirable optical property is fluorescence. In another preferred embodiment, the desirable optical property is fluorescence resonance energy transfer (“FRET”). In yet other preferred embodiment, the desirable optical property is phosphorescence.

Silanes useful for preparing the compositions of the present invention possess a leaving group capable of being displaced by an oxygen present in the metal oxide. Especially preferred leaving groups include C1-C4 alkoxides or —OH groups. In a preferred embodiment, the silane also comprises a reactive chemical group through which the stabilized nanoparticle may be bound to a molecule such as a protein, a nucleic acid, a lipid, a carbohydrate or another biological material such as a cell, a tissue sample or other similar materials. Especially preferred reactive chemical groups include primary amino groups, sulthydryl groups, aldehyde groups, carboxylate groups, alcohol groups, phosphate groups, ester groups and ether groups. Examples of preferred silanes comprising a reactive chemical group include Si(OH)ₙ(O(CH2)ₚCH₃)ₘ((CH₂)_{q}R), wherein 0≦n≦3; 0≦m≦3; 0≦p≦3; 0≦q≦10, n+m=3, and wherein R═H, halogen, OH, COOH, CHO, NH₂, COOR′, or OR′, (wherein R′ may be an alkyl or aryl moiety), SR″ (where R″ is H or a protecting group), or other commonly-used reagents in coupling chemistry. An example of a preferred silane comprising a sulfhydryl functional group (R=SH) is (3-mercaptopropyl)trimethoxysilane (SH(CH₂)₃Si(OCH₃)₃ available as Aldrich cat. no. 17561-7. Preferred silanes bearing a carboxyl functional group (R═COOH) can be prepared from preferred silanes bearing an amino functional group (R═NH₂) (such as, e.g., 3-aminopropyltrimethoxysilane (“APTMS”) H₂N(CH₂)₃Si(OCH₃)₃ (Sigma-Aldrich Chemicals, St. Louis, Mo.)) by reaction with succinic anhydride or glutaric anyhydride. An example of a preferred silane bearing an hydroxyl functional group (R═OH) is 3-glycidoxypropyltrimethoxysilane (Aldrich cat. no. 44016-7).

The invention also provides, in other preferred embodiments, for biological and other molecules derivatized with a metal oxide particle coated with a silane and having a desirable optical property. In one preferred embodiment, the biological molecule is a protein; in another it is a nucleic acid; in yet another it is a lipid; while in another it is a carbohydrate.

The invention also provides for direct assays to specifically detect the presence of an analyte in a sample, comprising specifically binding said analyte in said sample with a biological molecule derivatized with a metal oxide particle coated with a silane and having a desirable optical property, illuminating said particle bound to said analyte, and detecting said desirable optical property as a measure of the presence of said analyte in said sample. In one preferred embodiment, said desirable optical property is fluorescence. In another preferred embodiment, said desirable optical property is phosphorescence. In yet another preferred embodiment said desirable optical property is fluorescent resonance energy transfer (“FRET”). In preferred embodiments in which the metal oxide nanoparticle exhibits long phosphorescent or fluorescent lifetimes (such as, e.g., with lanthanide-containing nanoparticles), said desirable optical property is a fluorescence lifetime or a phosphorescent lifetime. In yet another preferred embodiment said biological molecule is selected from the group consisting of a protein, a nucleic acid, a lipid, and a carbohydrate.

In other preferred embodiments, the invention provides for indirect (i.e., competition) assays to specifically detect the presence of an analyte in a sample, comprising specifically binding an analyte ligand with a biological molecule derivatized with a metal oxide particle coated with a silane and having a desirable optical property, contacting said bound analyte ligand with a sample comprising an analyte capable of displacing said particle from said analyte ligand, illuminating said particle, and detecting said desirable optical property as a measure of the presence of said analyte in said sample. In one preferred embodiment, said desirable optical property is fluorescence. In another preferred embodiment, said desirable optical property is phosphorescence. In another preferred embodiment said desirable optical property is fluorescent resonance energy transfer (“FRET”). In yet another preferred embodiment said biological molecule is selected from the group consisting of a protein, a nucleic acid, a lipid, and a carbohydrate.

In yet another preferred embodiment, the invention provides for a method for coating a metal oxide particle having a desirable optical property with a silane having a leaving group capable of being displaced by an oxygen present in the metal oxide, comprising contacting said metal particle with said silane, and irradiating said metal particle and said silane with microwave radiation. In preferred embodiments, said silane comprises a chemical group capable of reacting with biological or other molecules. Especially preferred reactive chemical groups include primary amino groups, sulfhydryl groups, aldehyde groups, carboxylate groups, alcohol groups, phosphate groups, ester groups and ether groups.

The invention also provides for a method of derivatizing a molecule with a metal oxide particle coated with a silane having a chemical group capable of reacting with said molecule, said particle having a desired optical property, comprising contacting said particle with said molecule under conditions in which said chemical group reacts with said molecule. In preferred embodiments said molecule is a biological molecule selected from the group consisting of a protein, a nucleic acid, a lipid, and a carbohydrate. Especially preferred reactive chemical groups include primary amino groups, sulfhydryl groups, aldehyde groups, carboxylate groups, alcohol groups, phosphate groups, ester groups and ether groups.

### BRIEF DESCRIPTION OF THE DRAWINGS

FIG. 1. UV/V is absorbance spectrum (inset) and emission spectrum of Eu₂O₃ particles. The upper trace is the native particle spectrum, the lower trace is the 3-aminopropyltrimethoxysilane- (APTMS-) coated particle spectrum.

FIG. 2. Diagrams two bonding schemes (a) and (b) between a silane and a particle of Eu₂O₃.

FIG. 3. Fluorescence emission spectrum (a) and fluorescence image (b) of silane-coated Eu₂O₃ particles suspended in phosphate buffered saline.

FIG. 4. UVv is absorbance spectrum of Eu₂O₃ particle coated with 3-aminopropyltrimethoxysilane (APTMS) and reacted with ninhydrin to determine presence of free amino groups on particle surface.

FIG. 5. Scanning electron micrograph of Eu₂O₃ particles coated with silane.

FIG. 6. Derivatization of atrazine analog with 3-aminopropyltrimethoxysilane-(APTMS-) coated EU₂O₃.

FIG. 7. Fluorescence emission at 610 nm from triplicate measurements of atrazine analog labeled with Eu₂O₃ particle coated with 3-aminopropyltrimethoxysilane (APTMS) bound to anti-atrazine mouse monoclonal antibody, and captured with magnetic particle coated with goat anti-mouse IgG showing relationship between fluorescence intensity associated with magnetic particles and concentration of anti-atrazine mouse monoclonal antibody added to reaction mix.

FIG. 8. Competition immunoassay results. Fluorescence emission at 610 nm from triplicate measurements of atrazine analog labeled with Eu₂O₃ particle coated with 3-aminopropyltrimethoxysilane (APTMS) bound to anti-atrazine mouse monoclonal antibody, and captured with magnetic particle coated with goat anti-mouse IgG showing relationship between fluorescence intensity in solution phase and concentration of unlabeled atrazine added to reaction mix.

### DETAILED DESCRIPTION OF THE PREFERRED EMBODIMENTS

Stabilized nanoparticle compositions having desirable optical properties are provided by the instant invention, along with methods for their manufacture and use. In general, the nanoparticle compositions of the present invention comprise a metal oxide particle having a desirable optical property that has been coated with a silane. Preferred particle diameters are in the range of between about 10 and 1000 nm, more preferably between about 10 and 200 nm and even more preferably between about 10 and 100 nm, or between about 20 and 50 nm. In preferred embodiments, the metal oxide particles have the generic formula MeₓO_{y}, wherein 1≦x≦2, and 1≦y≦3, and wherein preferably, Me is a rare earth element selected from the lanthanide series and includes, but is not limited to, europium (Eu), cerium (Ce), neodymium (Nd), samarium (Sm), terbium (Tb), dysprosium (Dy), gadolinium (Gd), holmium (Ho), thulium (Tm), or Me may be chromium (Cr), yttrium (Y), iron (Fe). Other suitable metal oxide particles include silicon oxide (SiO₂), and aluminum oxide (Al₂O₃) mixed with Eu₂O₃ or Eu³⁺.

In other preferred embodiments, the metal oxide particle comprises a doped metal oxide particle by which is meant a metal oxide, and a dopant comprised of one or more rare earth elements. Suitable metal oxides include, but are not limited to, yttrium oxide (Y₂O₃), zirconium oxide (ZrO₂), zinc oxide (ZnO), copper oxide (CuO or Cu₂O), gadolinium oxide (Gd₂O₃), praseodymium oxide (Pr₂O₃), lanthanum oxide (La₂O₃), and alloys thereof. The rare earth element comprises an element selected from the lanthanide series and includes, but is not limited to, europium (Eu), cerium (Ce), neodymium (Nd), samarium (Sm), terbium (Tb), gadolinium (Gd), holmium (Ho), thulium (Tm), an oxide thereof, and a combination thereof. Nanoparticles of such oxides may be purchased from commercial suppliers or fabricated using methods known to those of ordinary skill in the art as set forth in, e.g., references 26 and 35, the disclosures of which are herein incorporated by reference.

The desirable optical properties of the compositions of the present invention include optical properties that allow the compositions to be useful as labeling agents, such as, e.g., fluorescence, fluorescence resonance energy transfer (“FRET”) , and phosphorescence. Thus, the compositions of the present invention may be used by one of skill in the art in the same manner as fluorescent dyes, FRET pairs and other labeling reagents, but with the advantages that nanoparticles bring to labeling technology in terms of larger Stokes shift, longer emission half-life (for lanthanide-containing nanoparticles), diminished emission bandwidth, and less photobleaching as compared with, e.g., traditional fluorescent dyes.

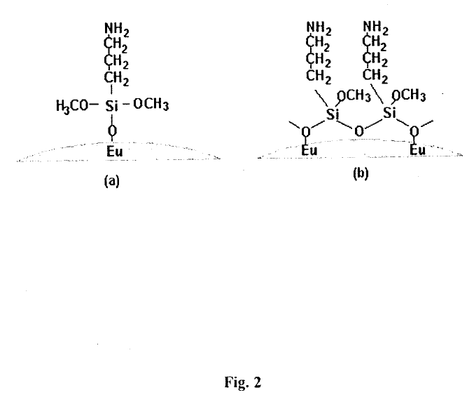

It has been surprisingly discovered by the inventors that nanoparticles having desirable optical properties may be chemically and physically stabilized by reacting the nanoparticles with a silane. The term “silane” refers to saturated silicon hydrides, analogues of the alkanes; i.e., compounds of the general formula SiₙH₂ₙ₊₂, and is intended to include silane, oligosilanes and polysilanes as well as hydrocarbyl derivatives and other derivatives. Silanes useful for the practice of the present invention have a leaving group capable of being displaced by an oxygen present in the metal oxide. Especially preferred leaving groups include C₁-C₄ alkoxides [or —OH group]. This reaction may be carried out by contacting a nanoparticle having a desirable optical property with a silane, and irradiating the reaction mixture with microwave radiation. Without wishing to be bound by theory, it is believed that oxide groups on the nanoparticle displace a leaving group on the silane to form structures such as are shown in FIGS. 2 (a) and (b). Preferred silanes include alkoxysilanes comprising such structures as —Si(OCH₃)₃, —Si(OC₂H₅)₃, —Si(OCH₃)H₂, —Si(OCH₃)(CH₃)₂, and —Si(OCH)₃)₂CH₃.

The stabilized silane-coated particles may be used as labeling reagents by exploiting a reactive chemical group attached to the silane. This group may be present on the silane at the time of initial reaction of the nanoparticle with the silane, or conveniently may be added at a later time using standard organic synthesis routes by which the reactive group is added to or is substituted for an existing group present on the silane. Methods for adding or substituting a reactive chemical group to a silane are well-known to those of skill in the art; representative examples of such methods may be found in, e.g., G. T. Hermanson, Bioconjugate Techniques (Academic Press, New York, 1996) and in D Gerion, F Pinaud, S C Williams, W J Parak, D Zanchet, S Weiss, A P Alivisatos, “Synthesis and Properties of Biocompatible Water-Soluble Silica-Coated CdSe/ZnS Semiconductor Quantum Dots,” J. Phys. Chem. B 2001, 105, 8861-8871, the entire disclosures of which are hereby incorporated by reference.

The nature of the reactive chemical group depends, of course, on the chemical nature of the target to be labeled using the compositions of the present invention. In preferred embodiments, the target to be labeled is a biological molecule such as a protein, a nucleic acid, a lipid or a carbohydrate. For these applications, silane-coated particles having desirable optical properties and suitable for labeling such a target conveniently may be prepared by reacting a particle having a desirable optical property with a silane having a primary amino group, a sulffiydryl group, an aldehyde group, a carboxylate group, an alcohol group, a phosphate group, or other reactive functional group. Methods of labeling biological molecules such as proteins, nucleic acids, lipids, and carbohydrates through reactive functional groups such as those that may be carried by the silane are well known in the art and are exemplified in, e.g., Functional Group Chemistry, J. R. Hanson & E. Abel, John Wiley & Sons, New Jersey (2002) ISBN: 0471224804, the entire disclosure of which is herein incorporated by reference. Examples of preferred silanes comprising a reactive functional group include Si(OH)ₙ(O(CH₂)ₚCH₃)ₘ((CH₂)_{q}R), wherein 0≦n≦3; 0≦m≦3; 0≦p<3; 0≦q≦10, n+m=3, and wherein R=H, halogen, OH, COOH, CHO, NH₂, PO₄, COOR′, or OR′, (wherein R′ may be an alkyl or aryl moiety), SR″ (where R″ is a protecting group on sulfur), or other commonly-used reagents in coupling chemistry. An especially preferred silane comprising a reactive functional group is 3-aminopropyltrimethoxysilane (“APTMS”) H₂N(CH₂)₃Si(OCH₃)₃, wherein with reference to the generic structure described above, n=0, p=0, m=3, q=3, and R=NH₂. An example of a preferred silane comprising a sulfhydryl functional group (R═SH) is (3-mercaptopropyl)trimethoxysilane (SH(CH₂)₃Si(OCH₃)₃ available as Aldrich cat. no. 17561-7. Preferred silanes bearing a carboxyl functional group (R═COOH) can be prepared from preferred silanes bearing an amino functional group (R═NH₂)(such as, e.g., 3-aminopropyltrimethoxysilane (“APTMS”) H₂N(CH₂)₃Si(OCH₃)₃ (Sigma-Aldrich Chemicals, St. Louis, Mo.)) by reaction with succinic anhydride or glutaric anyhydride. An example of a preferred silane bearing an hydroxyl functional group (R=OH) is 3-glycidoxypropyltrimethoxysilane (Aldrich cat. no. 44016-7).

Directly coating the particles with a silane having a reactive chemical group provides a simple way to preserve the desirable optical properties of the nanoparticles and simultaneously add to the particle a group that conveniently may be used to link the particle to a target molecule. Selection of the target molecule depends on the particular application. As one of ordinary skill will readily appreciate, the silane-coated particles may be used to label biological molecules to facilitate analyte detection using any type of assay that currently may be carried out using fluorescent, chemical, enzymatic, or radiolabeled molecules. These include hybridization assays, FRET assays, enzyme-linked immunosorbent assays (“ELISAs”), competition assays, or any other type of ligand binding assay known to one of skill in the art or developed at a later time that can be adapted for use with the compositions of the present invention. Examples of such assays may be found in references 5, 7, 9, 11, 12, 14-16, and 28-30.

In the working examples below, we describe one application of the invention using microwave chemistry to coat europium oxide nanoparticles with an amine-containing silane. Microwave absorption by lanthanides has been demonstrated and utilized in several applications [32, 33]. We have found silanes, such as 3-aminopropyltrimethoxysilane (“APTMS”), to be microwave transparent. Eu₂O₃ absorbs microwave energy, as a result of either an intrinsic property, or more likely, as a result of water or OH attached to the particle surfaces. As a result of this differential absorption in the particle suspension, heating can be confined to local regions without the need to heat the bulk solution. Reaction between the APTMS and the particle is concentrated at the particle surface. A reaction at the solid-liquid interface yields a layer of Si—O—(CH)ₓ—NH₂ that is covalently bound to the lanthanide oxide particles. Similar results can be achieved in a flow tube furnace with appropriate control of temperatures and time. Following treatment, the particles are used to label an organic molecule, an atrazine analog, which then is used in a competition immunoassay to detect unlabeled atrazine present in a sample.

### EXAMPLE 1

### Coating Eu₂O₃ Nanoparticles Using Microwave Chemistry

1 gram of Eu₂O₃ (99.99% purity, Sigma-Aldrich Chemicals, St. Louis, Mo.), was suspended in 50 mls of ethanol, and then sonicated for half an hour in a bath sonicator (Bransonic Model 52). The nanoparticle suspension was separated from larger Eu₂O₃ by low speed centrifugation (500×g at 4° C.), after which time the supernatant containing the suspension was removed from the low-speed pellet. Nanoparticles were collected by centrifuging the supernatant at 6000×g for 30 min. at 4° C. The pellet was collected and dried at 150° C. overnight.

Five mgs of the dried particles were added to a 10 ml beaker and approximately 1 ml of 3-aminopropyltrimethoxysilane (Sigma-Aldrich Chemicals, St. Louis, Mo.) was added to cover the Eu₂O₃ particles. The mixture was dispersed by bath sonication for 15 minutes using the same sonicator described above at the same power setting. It was then put onto the rotating stage in a microwave oven (Montgomery Ward, model LGS-1016A) operating at approximately 100 watts and emitting microwave radiation at a frequency of 2450MHz and irradiated for 2 minutes. It was removed from the microwave, stirred for about 2 minutes and bath sonicated again for 5 minutes. The procedure of heating and sonication was repeated between about 3 and 5 times until the suspension became a sticky mixture. The material was maintained at room temperature overnight, ground to a fine powder using a mortar and pestle and then heated at 150° C. for 30 minutes. The resulting particles were washed three times by suspending them in 50 mls of distilled water, then centrifuging the suspension at 6000×g for 30 minutes at room temperature. This procedure results in a chemical reaction at the surface of the Eu₂O₃ particles that is believed to covalently bond the silane to the Eu₂O₃ particles in either or both of the modes shown in FIG. 2.

### EXAMPLE 2

### Characterization of Eu₂O₃ -Silane Coated Particles

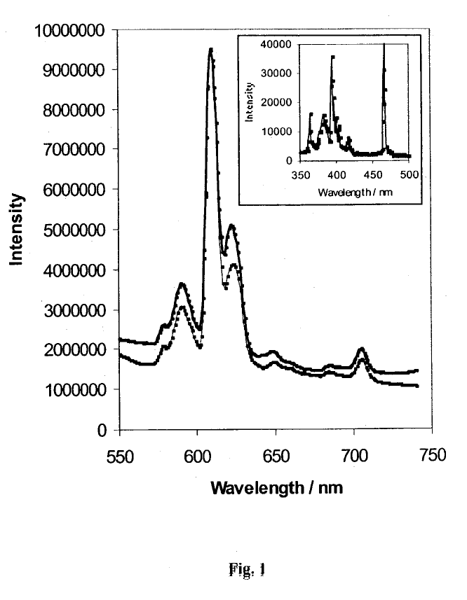

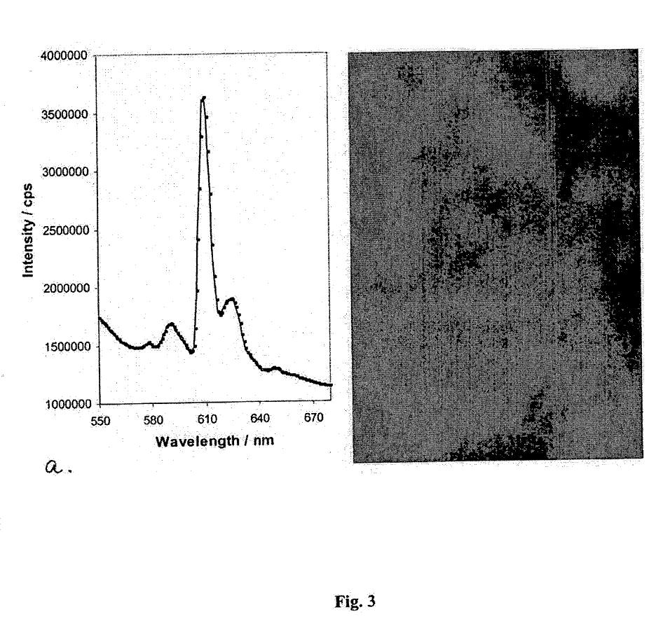

The resulting Eu₂O₃-silane coated particles were characterized using two different approaches. In the first approach, fluorospectrometry was used to characterize the fluorescence emission at 610 nm of the Eu₂O₃ particles before (FIG. 1) and after (FIG. 3(a)) silane coating following excitation at 394 nm or 466 nm. Particles were suspended in phosphate-buffered saline (“PBS”)(8 g L⁻¹ of NaCl, 1.15 g L⁻¹ of Na₂HPO₄, 0.2 g L⁻¹ of KCI, pH 7.4) to a final concentration of 1 mg/ml. 3 mls. of the suspension was loaded into a 4 cm quartz cuvette that then was placed inside a FluoroMax-2 spectrofluorometer (Instruments S. A., Inc., Edison, N.J.). Eu₂O₃ particles have a useful excitation region from about 356˜410 nm with a maximum at 394 nm. Another strong absorption is located at 466 nm. See FIG. 1 inset. The sample was excited with 466 nm light and the fluorescence emission spectrum determined. As illustrated in the FIG. 1 upper trace, a prominent peak centered at approximately 610 nm characterizes the emission spectrum of the uncoated Eu₂O₃ particles. The emission spectrum has the following salient characteristics, typical of europium and its chelates: (1) large Stokes shift (144 nm or 216 nm, depending on excitation wavelength); (2) a narrow, symmetric emission feature at 610 nm (full width half maximum, FWHM, or 8 nm); (3) a long life time (in this case measured with a time-resolved fluorescence system to be about 300 ms).

Most importantly, these characteristics were unchanged by the functionalization process. FIG. 3(a) illustrates that the silane-coated Eu₂O₃ particles have the same 610 nm emission peak as do the native particles. The emission spectra of the native Eu₂O₃ particles and those that have been functionalized with APTMS are overlaid in FIG. 1 to highlight the comparison. The upper trace is the native particle spectrum, the lower trace is the functionalized particle spectrum.

A narrow emission band from a fluorophore is potentially quite advantageous in bioanalysis. Considerable attention has been given to novel fluorophore labels in recent years, with a view to exploiting narrow spectral lines. For example, nanoscale quantum dots have an emission that is much narrower than conventional organic dyes. A typical functionalized, water soluble, nanocrystal of CdSe/ZnS in PBS was excited at 355 nm, leading to an emission peak at 533 nm with a 32 nm full width half maximum (FWHM) dispersion [34]. In comparison, the Eu₂O₃ nanoparticles exhibit a considerably narrower emission that will permit very selective detection against a large, spectrally broad background.

FIG. 3b shows a fluorescence micrograph of the silane-coated particles. An aliquot of a 0.01 mg/ml particle suspension in PBS was placed on a glass microscope slide, covered with a cover slip and imaged using a fluorescence microscope (Nikon microphot—Applied Scientific Instrumentation, Inc.). The coated Eu₂O₃ particles appear red following excitation by UV light. Overall magnification of the image in FIG. 3b is approximately 200x.

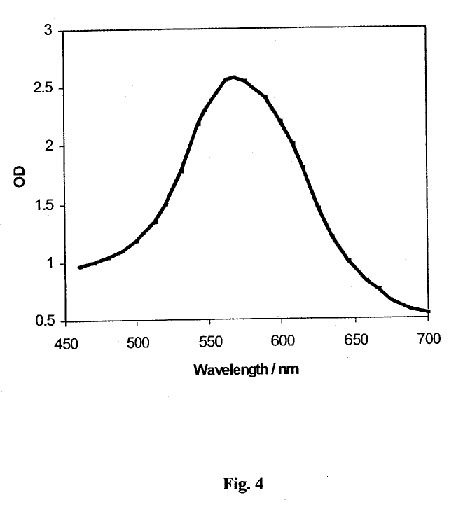

The particles also were chemically characterized using a ninhydrin test to determine the presence of —NH₂ groups on the surface of the particles. 100 μof a 0.8 mg/ml suspension of Eu₂O₃-silane coated particles in PBS was added to 0.5 ml of ethanol, and the resulting suspension was sonicated in a bath sonicator (Bransonic 52) for 20 minutes. 100 μof 0.2% ninhydrin (Sigma-Aldrich Chemicals, St. Louis, Mo.) in ethanol was added to the sonicated suspension and the reaction proceeded at room temperature for 10 minutes. In the presence of a primary amino group, ninhydrin generates a colored reaction product whose presence can be quantified using a spectrophotometer. The absorbance spectrum of an aliquot of the reaction product was determined using a UV/V is spectrometer (Cary 100 Bio) operating between 460 and 700 nm. (FIG. 4). The amount of reaction product may be quantified by measuring the optical density at 570 nm. As FIG. 4 illustrates, the Eu₂O₃ particles capped with silane and reacted with ninhydrin became blue (i.e., show an absorbance peak at 570 nm) as a result of the color change following reaction of ninhydrin and amino groups on the surface. The number of amine group on the surface of the europium particle (mol g⁻¹) can be evaluated by constructing a standard curve. We generated a standard curve using 3-aminopropyltrimethoxysilane in ethanol. The curve was fit using a linear least squares routine to yield Abs=−0.023+0.248 C with r=0.98, where Abs is the absorbance at 570 nm, and C is the concentration of amino groups. Using this standard curve, we determined that there are 6.3×10⁻⁶ mol NH₂/mg particles, or about 1280 amino groups on a 20 nm particle.

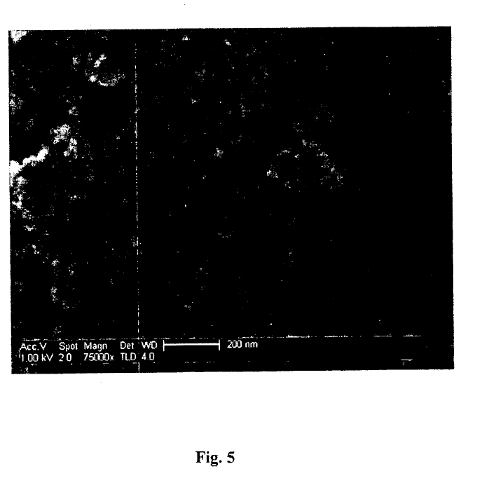

The particle size after silane capping also was characterized using scanning electron microscopy, which demonstrated that the particle sizes were in the range of around 100 nm to 200 nm (FIG. 5).

### EXAMPLE 3

### Atrazine Analog-Coniugated Eu₂O₃ Particles

Atrazine is a white, crystalline solid organic compound (M.W. 216) having the following structure.

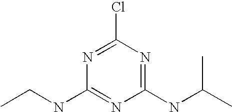

It is a widely-used herbicide for control of broadleaf and grassy weeds. Atrazine was estimated to be the most heavily used herbicide in the United States in 1987/89, with its most extensive use for corn and soybeans in Illinois, Indiana, Iowa, Kansas, Missouri, Nebraska, Ohio, Texas, and Wisconsin. Effective in 1993, its uses were greatly restricted.

The maximum contamination level goals (“MCLG”) for atrazine has been set by the Environmental Protection Agency (“EPA”) at 3 parts per billion (“ppb”) because EPA believes this level of protection would prevent the adverse health effects described below.

Short-term exposure to atrazine has been found to potentially cause the following health effects when people are exposed to it at levels above 3 ppb for relatively short periods of time: congestion of heart, lungs and kidneys; low blood pressure; muscle spasms; weight loss; damage to adrenal glands. Long-term atrazine exposure has the potential to cause the following effects from a lifetime exposure at levels above 3 ppb: weight loss, cardiovascular damage, retinal and some muscle degeneration; cancer.

EPA's Pesticides in Ground Water Database indicates numerous detections of atrazine at concentrations above the 3 ppb in ground water in several States, including Delaware, Illinois, Indiana, Iowa, Kansas, Michigan, Minnesota, Missouri, Nebraska and New York.

Because the ability to detect low concentrations (i.e. on the order of parts per billion) of atrazine is important for safeguarding drinking water supplies, we developed a competition immunoassay using an atrazine analog labeled with the Eu₂O₃ particles produced in Example 1 and characterized in Example 2 to demonstrate the use of these particles as labeling reagents.

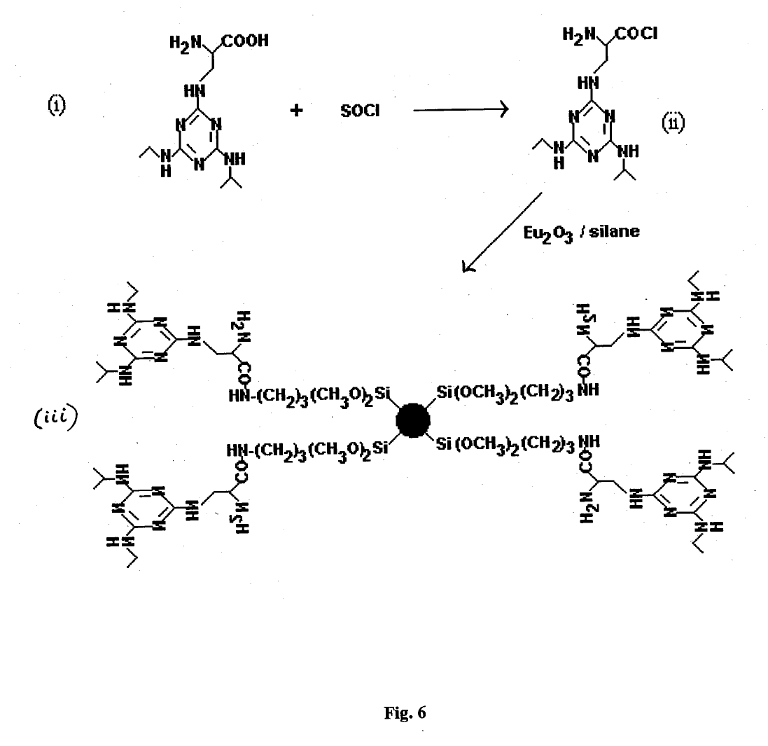

The derivatization of an atrazine analog with 3-aminopropyltrimethoxysilane-coated Eu₂O₃ was carried out as illustrated in FIG. 6 using the procedure described below:

Stock solution A: 18.4 mg of an atrazine analog (FIG. 6, structure “i”) was dissolved in 2.4 ml CHCl₃ with one drop of N,N-dimethylformamide (“DMF”) (Aldrich-Sigma Chemical Co., St. Louis, Mo.) in a 20 ml round bottom flask at 0-4° C., then 1.5 ml of thionyl chloride (SOCl₂) (Aldrich-Sigma Chemical Co., St. Louis, Mo.) was added dropwise over 5 min. The mixture was stirred and heated to 65-75° C. for 1 hour. The flask was put on a rotary evaporator to strip the solvent and excess thionyl chloride, resulting in a hazy oil of the acid chloride.

Stock solution B: 5 mg of the activated silane-coated Eu₂O₃ particles produced in Example 1 were suspended in 2 ml DMF and 15 mg of N,N-diisopropylethylamine (Aldrich-Sigma Chemical Co., St. Louis, Mo.) was added. The stock solutions A and B were mixed in a round bottom flask at −20° C. by using an iced saturated sodium chloride solution, and stirred overnight. The water temperature gradually increased to room temperature as the solution was stirred. The solvent was removed by evaporation, and the particles were washed by resuspending them in 15 mls of chloroform, and recovered by centrifuging the solution at 4° C. and 6000×g for 30 min.

After the atrazine analog-conjugated Eu₂O₃ particles were prepared, two procedures were used for particle characterization. First, fluorescence microscopy was used to observe the fluorescence image from the Eu₂O₃ particles and from the atrazine analog conjugated to the particles. Atrazine has a fluorescence emission peak at 430 nm following excitation at 345 nm. If the atrazine analog is conjugated to the Eu₂O₃ particles, a color intermediate between the 430 nm blue emission (atrazine) and the 610 nm red emission (Eu₂O₃) peaks of the two molecules should be observed (e.g., a green-blue color) under the fluorescence microscope when the sample is excited.

An aliquot of the atrazine-conjugated Eu₂O₃ particles was examined using a fluorescence microscope (Nikon Microphot—Applied Scientific Instrumentation, Inc.). As expected the particles appeared green-blue under these conditions, indicating that the atrazine analog had been successfully conjugated to the silane-coated Eu₂O₃ particles (data not shown). Upon filtering the light emitted by the particles to remove components having wavelengths shorter than 510 nm, the particles exhibited the expected red color characteristic of Eu₂O₃ fluorescence (data not shown).

Using another approach, a FluoroMax-2 spectrofluorometer (Instruments S.A., Inc., Edison, N.J.) was used to detect the fluorescence emission at 430 nm following excitation at 345 nm (corresponding to atrazine fluorescence), and the emission at 610 nm following excitation at 394 nm or 466 nm corresponding to Eu₂O₃ fluorescence). The spectrofluorometer results (not shown) confirmed the results obtained using fluorescence microscopy.

### EXAMPLE 4

### Competition Immunoassay using Atrazine Analog-Conjugated Eu₂O₃

To demonstrate that the atrazine analog-conjugated Eu₂O₃ particles could be bound by an anti-atrazine antibody, we carried out the following experiment using the atrazine analog-conjugated Eu₂O₃ particles prepared in Example 3.

The atrazine analog- Eu₂O₃ conjugate prepared in Example 3 was suspended in phosphate-buffered saline (“PBS”)(8 g L⁻¹ of NaCl, 1.15 g L⁻¹ of Na₂HPO₄, 0.2 g L⁻¹ of KCI, pH 7.4) to a final concentration of 25 μg ml⁻¹. A mouse monoclonal anti-atrazine antibody AM7B.2 [28]5.2 mg/ml protein stock solution was diluted 1:40 in PBSB (PBS containing 0.2% (w/v) bovine serum albumin (“BSA”)). A 100 μL aliquot of the atrazine analog—Eu₂O₃ conjugate in PBS, and 100 μL of various dilutions of the diluted AM7B.2 solution (sufficient to bring the final concentration of AM7B.2 in the 900 μL reaction mix to 0.25 μg/ml, 0.5 μg/ml, 0.75 μg/ml, and 1.0 μg/ml), were mixed in 12×75 mm borosilicate glass test tubes containing 700 μL of PBS and incubated for 15 minutes at 25° C. Following the incubation, 100 μL of magnetic particles coated with goat anti-mouse 1 gG (Polysciences Inc., Warrington, Pa.) was added to each test tube and the tubes were incubated for 30 minutes at room temperature with shaking. The particles then were separated from the supernatants by placing the tubes onto an Ohmicron 60 position Magnetic Rack (Strategic Diagnostics Inc., Newark, Del.). The particles were washed with 1 ml of PBS. The supernatants from each tube initially recovered along with those from the wash were individually pooled (total 2 ml for each tube) and the magnetic beads from each tube were suspended in 1 ml of PBS. The fluorescence intensity in both supernatant and beads was measured using the spectrofluorometer (Instruments S.A., Inc., Edison, N.J.) with excitation at 394 nm or 466 nm and emission at 610 nm.

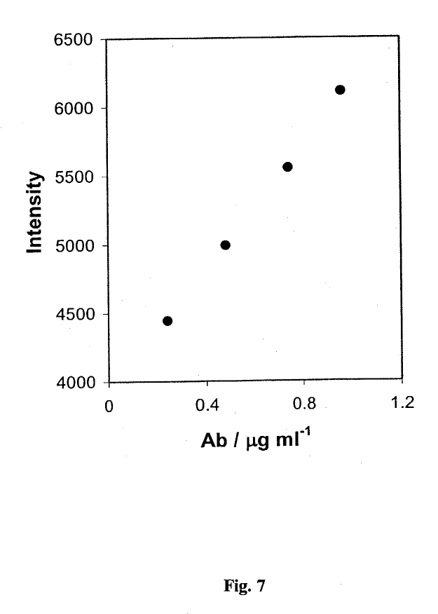

The results of this experiment are illustrated in FIG. 7. which shows a linear relationship between the binding of the atrazine analog-conjugated Eu₂O₃ particles (as indicated by the intensity of the fluorescence emission at 610 nm of the recovered magnetic particles) and the concentration of the AM7B.2 anti-atrazine monoclonal antibody added to the reaction mix. Three measurements were taken for every point and the standard error was 5%. We next carried out the competition experiment described below.

The atrazine analog- Eu₂O₃ conjugate prepared in Example 3 was suspended in phosphate-buffered saline (“PBS”)(8 g L⁻¹ of NaCl, 1.15 g L⁻¹ of Na₂HPO₄, 0.2 g L⁻¹ of KCI, pH 7.4) to a final concentration of 25 μg ml⁻¹. The mouse monoclonal anti-atrazine antibody AM7B.2 [28] stock solution at 5.2 mg/ml protein was diluted 1:40 in PBSB (PBS containing 0.2% (w/v) bovine serum albumin (“BSA”)). A 100 μL aliquot of the atrazine analog- EU₂O₃ conjugate in PBS, 100 μL of AM7B.2 (0.1 nmol), and 100 μL of different dilutions (9.4 nmol˜94 mmol) of unlabeled atrazine were mixed in 12×75 mm borosilicate glass test tubes containing 600 μL of PBS and incubated for 15 minutes at 25° C. Following the incubation, 100 μL of magnetic particles coated with goat anti-mouse 1 gG (Polysciences Inc., Warrington, Pa.) was added to each test tube and the tubes were incubated for 30 minutes at room temperature with shaking. The particles then were separated from the supernatants by placing the tubes onto an Ohmicron 60 position Magnetic Rack (Strategic Diagnostics Inc., Newark, Del.). The particles were washed with 1 ml of PBS. The supernatants from each tube initially recovered along with those from the wash were individually pooled (total 2 ml for each tube) and the magnetic beads from each tube were suspended in 1 ml of PBS. The fluorescence intensity in both supernatant and beads was measured using the spectrofluorometer (Instruments S.A., Inc., Edison, N.J.) with excitation at 394 nm or 466 nm and emission at 610 nm.

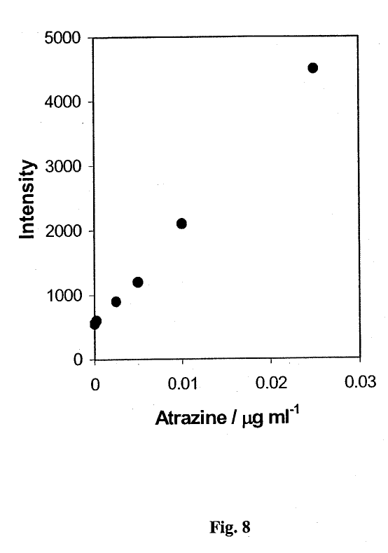

The results of the competition experiment demonstrated that the unlabeled atrazine and atrazine-conjugated Eu₂O₃ reacted competitively with the AM7B.2 antibody. As expected, the amount of atrazine-conjugated Eu₂O₃ bound to the magnetic beads was found to decrease with increasing concentrations of unlabeled atrazine added to the reaction solution (data not shown). At the same time, the amount of atrazine-conjugated Eu₂O₃ found in the supernatant increased with increasing concentrations of unlabeled atrazine, as shown in FIG. 8 which shows the fluorescence intensity at 610 nm in the recovered supernatants as a function of unlabeled atrazine concentration (triplicate measurements, standard error 4.5%). The detection limit of this assay was about 0.1 nM equal to 0.02 ppb.

References

1. Lim, M. J., Patton, W. F., Lopez, M. F., Spofford, K. H., Shojaee, N., Shepro, D. A. “Luminescent Europium Complex for the Sensitive Detection of Proteins and Nucleic Acids Immobilized on Membrane Supports,” Anal Biochem., (Feb. 15, 1997) 245:2, 184-195

2. Liu, X. J., Li, Y. Z., Ci, Y. X., “Time-resolved Fluorescence Studies of the Interaction of the Eu3+ Complexes of Tetracycline Analogues with DNA,” Anal Chim Acta., (1997) 345, 213-217.

3. Mathis, G. Rare, “Earth Cryptates and Homogeneous Fluoroimmunoassays with Human Sera,” Clin. Chem., (1993) 39, 1953-1959.

4. Saha, A. K., Kross, K., Kloszewski, E. D., Upson, D. A., Toner, J. L., Snow, R. A., Black, C. D. V., Desai, V. C., “Time-resolved Fluorescence of a New Europium-chelate Complex: Demonstration of Highly, Sensitive Detection of Protein and DNA Samples,” J. Am. Chem. Soc., (1993) 115, 11032-11033.

5. Horiguchi, D., Sasamoto, K., Terasawa, H., Mochizuki, H., Ohkura, Y., “A Novel Time-Resolved Fluoroimmunoassay Using a Macrocyclic Europium Ligand as a Label,” Chem. Pharm. Bull., (1994) 42, 972-975.

6. Yuan J. L., Wang G. L., Majima K., Matsumoto K., “Synthesis of a Terbium Fluorescent Chelate and its Application to Time-resolved Fluoroimmunoassay,” Anal Chem., (2001) 73, 8, 1869-1876,

7. Dickson, E. F., Pollak, A., Diamandis, E. P., “Ultrasensitive Bioanalytical Assays Using Time-resolved Fluorescence Detection,” Pharmacol. Ther., (1995) 66, 207-235.

8. Scorilas, A., Bjartell, A., Lilja, H., Moller, C., “Diamandis EP Streptavidin-polyvinylamine Conjugates Labeled with a Europium Chelate: Applications in Immunoassay, Immunohistochemistry, and Microarrays,” Clini Chem., (2000) 46:9, 1450-1455.

9. Matsumoto, K., Yuan, J. G., Wang, G. L., Kimura, H., “Simultaneous Determination of Alpha-Fetoprotein and Carcinoembryonic Antigen in Human Serum by Time-resolved Fluoroimmunoassay,” Anal Biochem., (1999) 276:1, 81-87, 1.

10. Yuan, J. L., Matsumoto, K., Kimura, H., “A New Tetradentate Beta-diketonate-Europium Chelate That Can Be Covalently Bound to Proteins for Time-resolved Fluoroimmunoasaay,” Anal Chem., (1998) 70:3, 596-601.

11. Yuan, J. L., Wang, G. L., Kimura, H., Matsumoto, K., “Highly Sensitive Time-resolved Fluoroimmunoassay of Human Immunoglobulin E by Using A New Europium Fluorescent Chelate as a Label Fluoroimmunoassay,” Anal Biochem., (1997) 254:2, 283-287.

12. Rulli, M., Kuusisto A., Salo J., Kojola H., Simell O., “Time-Resolved Fluorescence Imaging in Islet Cell Autoantibody Quantitation,” J. Immunol Methods, (1997) 208:2, 169-179.

13. Ci, Y. X., Yang, X. D., Chang, W. B., “Fluorescence Labeling with Europium Chelate of Beta-diketones and Application in Time-Resolve Fluorescence Immunoassay,” (TR-FIA) (1995) J. Immunol Methods, 179:2, 233-241.

14. Yang, X. D., Ci, Y. X., Chang, W. B., “Time-resolved Fluorescence Immunoassay with Measurement of a Europium Chelate in Solution-Dissociation Conditions and Application for Determination of Cortisol,” Anal Chem., (1994) 66:15, 2590-2594.

15. Vaisanen, V., Harma, H., Lilja, H., Bjartell, A., “Time-resolved Fluorescence Imaging for Quantitative Histochemistry Using Lanthanide Chelates in Nanoparticles and Conjugated to Monoclonal Antibodies,” Luminescence, (2000) 15:6, 389-397.

16. Qin, Q.P., Lovgren, T., Pettersson, K., “Development of Highly Fluorescent Detection Reagents for the Construction of Ultrasensitive Immunoassays,” Anal Biochem., (Apr. 1, 2001) 73:7, 1521-1529.

17. Bruchez, M. Jr., Moronne, M., Gin, P., Weiss, S., Alivisatos, A. P., “Semiconductor Nanocrystals As Fluorescence Biological Labels,” Science, (1998) 281, 2013-2015.

18. Warren, C. W., Chan, W., Nie, S., Quantum Dot Bioconjugates for Ultrasensitive Nonisotopic Detection, Science (1998) 281, 2016-2018.

19. Han, M. Y., Gao, X. H., Su, J. Z., Nie, S., “Quantum-dot-tagged Micro beads for Multiplexed Optical Coding of Biomolecules,” Nature Biotechnology, (2001) 19:7, 631-635.

20. Karmic, “Biologists Join the Dots,” Nature, (Oct. 4, 2001) 413:6855, 450-452.

21. Casco, T., “DNA's New Colors,” Nature Reviews Genetics, (2001) 2, 567-567.

22. Rosenthal, V S., “Bar-coding Biomolecules with Fluorescent Nanocrystals,” Nature Biotechnology, (2001) 19, 621-622.

23. Mitosis, H., Mauro, J. M., Goldman, EAR, Green, T M., Anderson, GAP., Sunder, V S., Basenji, M. G., “Bioconjugation of Highly Luminescent Colloidal CdSe-ZnS Quantum Dots with an Engineered Two-domain Recombinant Protein,” Physica Status Solidi B-Basic Research, (2001) 224, 277-283.

24. Mattoussi, H., Mauro, J. M., Goldman, E. R., Anderson, G. P., Sundar, V. C., Mikulec, F. V., Bawendi, M. G., “Self-assembly of CdSe-ZnS Quantum Dot Bioconjugates Using an Engineered Recombinant Protein,” J. Am. Chem. Soc (2000) 122, 12142-12150.

25. Nogami, M., Abe, Y., “Properties of Sol-gel-derived A1203-SiO2 Glasses Using Eu³⁺ Ion Fluorescence Spectra,” J. Non-Cryst Solids, (1996) 197, 73.

26. Patra, A., Sominska, E., Ramesh, S., Koltypin, Yu., Zhong, Z., Minti, H., Reisfeld, R., and Gedanken, A., “Sonochemical Preparation and Characterization of Eu₂O₃ and Tb₂O₃ Doped in and Coated on Silica and Alumina Nanoparticles,” J. Phys. Chem., (1999) 103, 3361 -3365.

27. Horrocks, W. D. Jr., Sudnick, D. R., “Lanthanide Ion Luminescence Probes of the Structure of Biological Macromolecules,” Acc. Chem. Res,. (1981) 14, 384.

28. Karu, A. E.; Harrison, R. O; Schmidt, D. J.; Clarkson, C. E.; Grassman, J.; Goodrow, M. H.; Lucas, A.; Hammock, B. D.; White, R. J.; Van Emon, J. M., “Monoclonal immunoassay of trizine herbicides: development and implementation.” In Immunoassays for Trace Chemical Analysis: Monitoring Toxic Chemicals in Humans, Food, and Environment, Vanderlaan, M., Stanker, L. H., Watkins, B. E., Roberts, D. W., (Eds.); ACS Symposium Series 451; American Chemical Society: Washington, D.C., (1991), 59-77.

29. Goodrow, M. H.; Sanborn, J. R.; Stoutamire, D. W.; Gee, S. J.; Hammock, B. D., “Strategies for immunoassay hapten design. in Immunoanalysis of Agrochemicals: Emerging Technologies.” Nelson, J. O., Karu, A. E. and Wong, R. B., (Eds.) American Chemical Society Symposium No. 586, American Chemical Society, Washington, D.C., (1995), 1-19.

30. Lucas, A. D.; Jones, A. D.; Goodrow, M. H.; Saiz, S. G.; Blewett, C.; Seiber, J. N.; and Hammock, B. D., “Determination of atrazine metabolites in human urine: Development of a biomarker of exposure,” Chem. Res. Toxicol. (1993) 6, 107-115.

31. Slavik, J. Fluorescent Probes in Cellular and Molecular Biology; CRC Press: Boca Raton, Fla., 1994.

32. Sen Gupta, J. G.; Bertrand, N. B. Talanta 1995, 42, 1595 - 1607.

33. Haque, K. E. Int. J. Miner. Process. 1999, 57, 1-24.

34. Bruchez, M.; Moronne, M.; Gin, P.; Weiss, S.; Alivisatos, A. P. Science 1998, 281, 2013 2016.

35. Leppert, V. J., Harvey, A. S., et al., Paper 4809-49 SPIE 45th Annual Meeting, Seattle Wash., July 2002.

The foregoing description of the embodiments of the invention has been presented for the purposes of illustration and description. It is not intended to be exhaustive or to limit the invention to the precise forms disclosed. Persons skilled in the relevant art can appreciate that many modifications and variations are possible in light of the above teaching. Concentrations, sizes and other parameters stated in the specification and the claims are for example only and are intended to include variations consistent with the practice of the present invention. Such permissible variations are readily determined by persons of skill in the art in light of the instant disclosure and typically encompass between about ±10% to about ±20% of the stated parameter. It is therefore intended that the scope of the invention be limited not by this detailed description, but rather by the claims appended hereto. References to publications, patent applications and issued patents contained in this specification are herein incorporated by reference in their entirety for all purposes.

We claim:

1. A composition, comprising:
a silane-coated metal oxide nanoparticle, wherein said metal oxide has a generic formula MeₓO_{y}, wherein Me is a metal, 1≦x≦2 and 1≦y≦3, and wherein said nanoparticle is capable of light emission.

2. The composition of claim 1, wherein said nanoparticle is capable of fluorescent light emission.

3. The composition of claim 1, wherein said nanoparticle is capable of phosphorescent light emission.

4. The composition of claim 1, wherein the diameter of said nanoparticle is between 10 nm and 100 nm.

5. The composition of claim 4, wherein the diameter of said nanoparticle is between 10 nm and 200 nm.

6. The composition of claim 5, wherein the diameter of said nanoparticle is between 10 nm and 100 nm.

7. The composition of claim 6, wherein the diameter of said nanoparticle is between 20 nm and 50 nm.

8. The composition of claim 1, wherein Me is a rare earth element.

9. The composition of claim 8, wherein said rare earth element is a lanthanide.

10. The composition of claim 9, wherein said lanthanide an element selected from the group consisting of Eu, Ce, Nd, Sm, Tb, Dy, Gd, Ho, and Tm.

11. The composition of claim 10, wherein said lanthanide is Eu and said metal oxide is Eu₂O₃.

12. The composition of claim 10, wherein said lanthanide is Tb and said metal oxide is Tb₂O₃.

13. The composition of claim 1, wherein x=2 and y=3.

14. The composition of claim 1, wherein said metal oxide is selected from the group consisting of Cr₂O₃, Y₂O₃, Fe₂O₃, and Fe₃O₄.

15. The composition of claim 1, wherein said metal oxide is selected from the group consisting of SiO₂, Al₂O₃, TiO₂, and ZrO₂, and wherein said nanoparticle further comprises Eu₂O₃ or Eu³⁺.

16. The composition of claim 1, wherein said metal oxide nanoparticle comprises a metal oxide and a rare earth element dopant.

17. The composition of claim 16, wherein said metal oxide is selected from the group consisting of Y₂O₃, ZrO₂, ZnO, CuO, Cu₂O₃, Gd₂O₃, Pr₂O₃, and La₂O₃, and said rare earth element dopant is a lanthanide selected from the group consisting of Eu, Ce, Nd, Sm, Tb, Dy, Gd, Ho, and Th.

18. The composition of claim 1, wherein said silane has generic formula Si(OH)ₙ(O(CH₂)ₚCH₃)ₘ((CH₂)_{q}R), wherein 0≦n≦3; 0≦m≦3; 0≦p≦3; 0≦q≦10, n+m=3, and wherein R═H, halogen, OH, COOH, CHO, NH₂, PO₄, COOR′, OR′ and R′ is an alkyl or aryl moiety, or R═SR′ and R′ is H or a protecting group.

19. The composition of claim 1, further comprising a protein molecule covalently bound to said silane.

20. The composition of claim 19, wherein said protein is an antibody.

21. The composition of claim 1, further comprising a nucleic acid molecule covalently bound to said silane.

22. The composition of claim 1, further comprising a lipid molecule covalently bound to said silane.

23. The composition of claim 1, further comprising a carbohydrate molecule covalently bound to said silane.

24. An improved assay for detecting the presence of an analyte in a sample, said assay comprising contacting a sample suspected of containing said analyte with a labeled composition under conditions in which said analyte specifically binds said labeled composition, and detecting the presence of said label, the improvement comprising: using the composition of claim 19, claim 20, claim 21, claim 22, or claim 23 as said labeled composition.

25. The assay of claim 24, wherein said assay is a direct assay.

26. The assay of claim 24, wherein said assay is a competition assay.

27. A method for coating a metal oxide nanoparticle with a silane, comprising: preparing a solution of said metal oxide nanoparticle and said silane, and irradiating said solution with microwave radiation.

28. The method of claim 27, wherein said metal oxide is Eu₂O₃.

29. The method of claim 28, wherein said silane is 3-aminopropyltrimethoxysilane.

30. The method of claim 27, wherein said metal oxide is Tb₂O₃.

31. The method of claim 30, wherein said silane is 3-aminopropyltrimethoxysilane.

32. An improved method for labeling a molecule, said method comprising reacting said molecule with a label, wherein said label has a reactive chemical group capable of reacting with said molecule, the improvement comprising: using the composition of claim 18 as said label.

33. The method of claim 32, wherein said molecule is a protein molecule.

34. The method of claim 33, wherein said protein is an antibody.

35. The method of claim 32, wherein said molecule is a nucleic acid molecule.

36. The method of claim 32, wherein said molecule is a lipid molecule.

37. The method of claim 32, wherein said molecule is a carbohydrate molecule.
